(19) United States
(12) Patent Application Publication
(10) Pub. No.: US 2024/0421842 A1
(43) Pub. Date: Dec. 19, 2024

(54) TRANSMISSION POWER MANAGEMENT WITH PARAMETERIZED ASSIGNED CLASSIFICATION FILTERS

(71) Applicant: Microsoft Technology Licensing, LLC, Redmond, WA (US)

(72) Inventor: Charbel KHAWAND, Sammamish, WA (US)

(21) Appl. No.: 18/334,751

(22) Filed: Jun. 14, 2023

(51) Int. Cl. H04B 1/3827; G01P 13/00; H04W 52/28

(52) U.S. Cl. CPC H04B 1/3838; G01P 13/00; H04W 52/28

(57) ABSTRACT

Transmission power of a communication device is managed by detecting motion of the communication device using a motion sensor, classifying detected motion according to one or more motion classification conditions, assigning one or more motion classification filters to monitor the detected motion, setting one or more filter parameters for the one or more motion classification filters, a monitoring the detected motion using the one or more filter parameters.

### RELATED APPLICATIONS

This matter relates to U.S. Pat. No. 11,490,338 issued on Nov. 1, 2022 entitled MOTION-RESPONSIVE TRANSMISSION POWER MANAGEMENT, the entirety of which is incorporated by reference.

### BACKGROUND

Communication devices emit electromagnetic radio frequency signals to transmit and receive data. Safety concerns about absorbed radiation from the radio frequency signals transmitted have led regulatory bodies, such as the U.S. Federal Communications Commission, to set Specific Absorption Rate (SAR) regulations limiting the amount of radiation absorbed by a user of a communication device. Compliance with SAR regulations often involves reducing the transmission power of communication antennas in the communication device, which generally reduces transmission performance.

### SUMMARY

The described technology provides implementations of systems and methods for power management in a communication device. More specifically, the described technology provides implementations of systems and methods for SAR-related power management in a communication device.

In some aspects, the techniques described herein relate to a method of monitoring detected motion of a communication device, including: detecting motion of the communication device using a motion sensor; classifying detected motion according to one or more classification conditions; assigning one or more motion classification filters to monitor the detected motion; setting one or more filter parameters for the one or more motion classification filters; and monitoring the detected motion using the one or more filter parameters.

In some aspects, the techniques described herein relate to a system having a processor and a memory for monitoring detected motion of a communication device of a communication device, including: a motion sensor to detect motion of the communication device; a motion detection module executable by the processor to classify detected motion according to one or more motion classification conditions; and one or more motion classification filters to monitor the detected motion using one or more filter parameters set for the one or more motion classification filters by the motion detection module.

In some aspects, the techniques described herein relate to one or more tangible processor-readable storage media embodied with instructions for executing on one or more processors and circuits a process for monitoring detected motion of a communication device, including: detecting motion of the communication device using a motion sensor; classifying detected motion according to one or more classification conditions; assigning one or more motion classification filters to monitor the detected motion; setting one or more filter parameters for the one or more motion classification filters; and monitoring the detected motion using the one or more filter parameters.

This summary is provided to introduce a selection of concepts in a simplified form that is further described below in the Detailed Description. This summary is not intended to identify key features or essential features of the claimed subject matter, nor is it intended to be used to limit the scope of the claimed subject matter.

Other implementations are also described and recited herein.

### BRIEF DESCRIPTION OF THE DRAWINGS

FIG. 1 illustrates an example use case for a communication device.

FIG. 2 illustrates an example communication device.

FIG. 3 illustrates a schematic view of an example communication device.

FIG. 4 illustrates an example signal of a motion sensor.

FIG. 5 illustrates example operations for using a plurality of motion sensors in a communication device.

FIG. 6 illustrates an example communication device for implementing the features and operations of the described technology.

### DETAILED DESCRIPTIONS

Consumer electronic devices may be equipped with wireless communication circuitry emitting radio frequency (RF) electromagnetic fields that can be absorbed by human tissue positioned in proximity to the wireless communication circuitry. For example, the wireless communications circuitry may transmit and receive RF signals in mobile telephone RF bands, LTE RF bands, Wi-Fi network RF bands, and GPS RF bands. To protect humans from potentially harmful levels of RF radiation when using such devices, it may be beneficial to reduce the transmission power of RF transmitters for communication devices when in close proximity to a user. In addition, regulatory agencies have imposed regulations limiting RF transmission power from some wireless electronic devices, such as tablet computers and mobile phones. While reducing transmitted RF signal strength may enhance user safety and/or compliance with safety regulations, significant reductions in the transmitted carrier signal strength can result in decreased device communication performance, including without limitation dropped connections (e.g., a dropped call) and/or delays in the transmission of information.

One approach to determining when a user is interacting with a communication device such that the RF transmission power levels may be reduced is based on detecting and classifying motion of the communication device. For example, detected motion of a communication device can indicate that a user is interacting with (e.g., actively or passively contacting) the communication device and allow for the device to perform a SAR motion backoff when in a SAR IN MOTION state. However, a considerable amount of device motion can be attributable to other sources of background motion, such as motion of internal communication device componentry (e.g., a fan), motion of a surface on which a communication device rests (e.g., in a moving vehicle), ambient vibrations an environment in which the communication device is located (e.g., music), etc. When detecting background motion without human interaction motion, the communication device preferably does not trigger a SAR motion backoff when in a SAR STATIONARY state. As such, when determining whether to place a communication device in a SAR IN MOTION state, it may be useful to discern motion related to human activity from background motion.

Discriminating between background motion and human interaction motion can be difficult. Some approaches to discerning between human interaction motion and background motion may include relatively sophisticated processing of a signal representative of the motion of the communication device. The motion signal may be analyzed to classify detected motion that relates either to human activity or other motion. For example, application of artificial intelligence techniques or advanced signal processing techniques such as performing fast Fourier transformations (FFTs) may be used for classification of motion into human activity (in which a SAR IN MOTION state is preferably active) or background motion (in which a SAR STATIONARY state is active) While such approaches may allow for discerning between human interaction motion and background motion, processing a motion signal from a motion sensor of a communication device using these techniques may be relatively resource-intensive when performing such processing. In turn, computational bandwidth and power consumption may be increased when performing such signal processing to discern between human interaction motion and background motion.

The present technology may utilize one or more motion classification filters that may be assigned to monitor a detected motion that has been classified to a particular SAR motion state. Classification of detected motion may include comparisons, for example, between detected motions and one or more of thresholds or motion profiles, application of an artificial intelligence model, classification based on a frequency domain representation of the signal (e.g., application of an FFT to the signal), or other approaches that allows classification of the detected motion between either SAR IN MOTION or SAR STATIONARY states. As noted above, such classification conditions may be determined according to any one or more of a plurality of techniques that may be relatively resource intensive.

As such, once a classification condition is identified, one or more of the motion classification filters may be assigned to monitor the detected motion associated with the classification condition. Such classification filters may be hardware and/or software filters that may be much less resource intensive to execute than the components used to classify the motion. The one or more motion classification filters may have one or more filter parameters set for the motion classification filters to allow the one or more classification filters to monitor the detected motion without requiring continued execution of a resource-intensive motion detection module. The one or more motion classification filters may therefore continue to monitor the detected motion associated with a classification condition to maintain the communication device at an appropriate power transmission level associated with the classification condition. In the event the detected motion is no longer detected, the communication device may readjust the level and/or the motion detection module may re-execute to determine if another classification condition exists in the motion sensor data.

Use of assigned motion classification filters that are parameterized for a given detected motion using filter parameters may also be more versatile and robust than use of static filters. For instance, a motion model that characterizes motion related to user interaction may be adaptable upon use of the device such that predetermined filter parameters may not be effective prior to detection and classification of such motion. Furthermore, the variability of potential background motion sources may not allow fixed filter parameters to be effective. Also, some predictable background motion (e.g., vibration of a cooling fan) may change over time such that even initial fixed filter parameters may not be effective over the life of the communication device.

Accordingly, use of the motion classification filters according to the technology described herein may allow for an adaptable approach to monitor classified motion in a resource-efficient manner. The approach may allow for leveraging the benefit of sophisticated classification approaches to classify motion in an adaptable manner and thereafter assigning the motion classification filter with appropriate filter parameters to monitor the classified motion in a more efficient manner.

The present disclosure describes use of motion classification filters for monitoring classified detected motion for purposes of SAR-related power management. However, it may be appreciated that the approach described herein may be used to monitor any detected and/or classified motion. For example, a motion sensor may monitor and classify detected motion associated with a health of a hardware component of the communication device such as a fan. In this regard, a condition (e.g., fan failure) may be detected based on a motion signal for a motion sensor and the condition may be monitored using motion classification filters. Thus, the motion classification filters may be an efficient way to provide monitoring of any classified detected motion, including use in SAR-related power management.

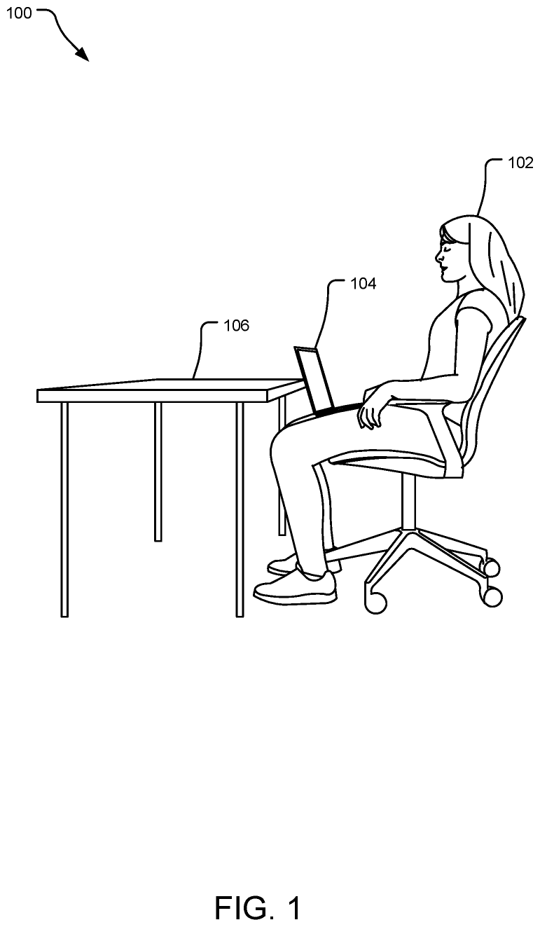

FIG. 1 illustrates an example environment 100 in which motion of a communication device 104 may be used to classify motion of the communication device 104 for SAR-related power management of the communication device 104. Specifically, a transmission power of an RF transmitter of the communication device 104 may be adjusted based on the classification of detected motion of the communication device 104 in a SAR-related power management scheme. In this regard, detected motion classified as a SAR IN MOTION state may result in the transmission power of the RF transmitter being reduced to facilitate improved user safety. Detected motion classified as SAR STATIONARY state may allow the transmission power of an RF transmitter to remain unaffected at a full operational power to promote device performance. As will be shown below, classification of detected motion may be complicated by subtle user interactions and environmental motion experienced by a communication device 104.

The environment 100 of FIG. 1 shows the communication device 104, an inanimate surface 106, and a user 102. The inanimate surface 106 is shown as a table on which the communication device 104 may be placed (although not depicted as such in FIG. 1). In cases where the communication device 104 rests on the inanimate surface 106 and the user 102 is not engaged with the communication device 104, a motion sensor of the communication device 104 may not detect motion indicative of user interaction with the communication device 104. However, background motion may still be detected by the motion sensor of the communication device 104, even when simply resting on the inanimate surface 106. For example, ambient vibrations such as those induced by music, mechanical vibrations, or other ambient vibrations may still be detected by the motion sensor of the communication device 104. However, such background motion may be discerned by a classifier as background motion not attributable to human interaction with the communication device 104 such that the classifier of a controller may classify the detected motion as a SAR STATIONARY state.

In another use case, the communication device 104 may not rest on the inanimate surface but on a lap of the user 102. Accordingly, despite the fact that the user 102 is not directly engaged with the communication device 104 (e.g., the user is not typing or scrolling), communication device 104 may be in sufficient contact with the user 102 such that a SAR IN MOTION state is preferably triggered. Classifying detected motion when the communication device 104 is in contact with, but not actively engaged by, the user 102 may be particularly difficult to discern. For example, the detected motion may primarily correspond to subtle movements such as those imparted by the respiration, heartbeat, or other low amplitude movements of the user 102. Discerning these subtle movements from background motion may employ relatively sophisticated models that utilize relatively intensive computational resources to execute. However, in the use case shown in FIG. 1, it may still be possible to determine that the communication device 104 is in a SAR IN MOTION state despite passively resting on the lap of the user 102.

However, once it is determined that the communication device 104 is in the SAR IN MOTION state, continued monitoring of such motion by the relatively resource-intensive processes used to determine the SAR IN MOTION classification may contribute to a degradation in performance and/or battery performance of the communication device 104. As such, as described in greater detail herein, the technology of the present disclosure may allow one or more motion classification filters to be assigned to monitor detected motion that has been classified. In turn, the motion classification filter may confirm the continued detected motion as classified using more resource-efficient approaches.

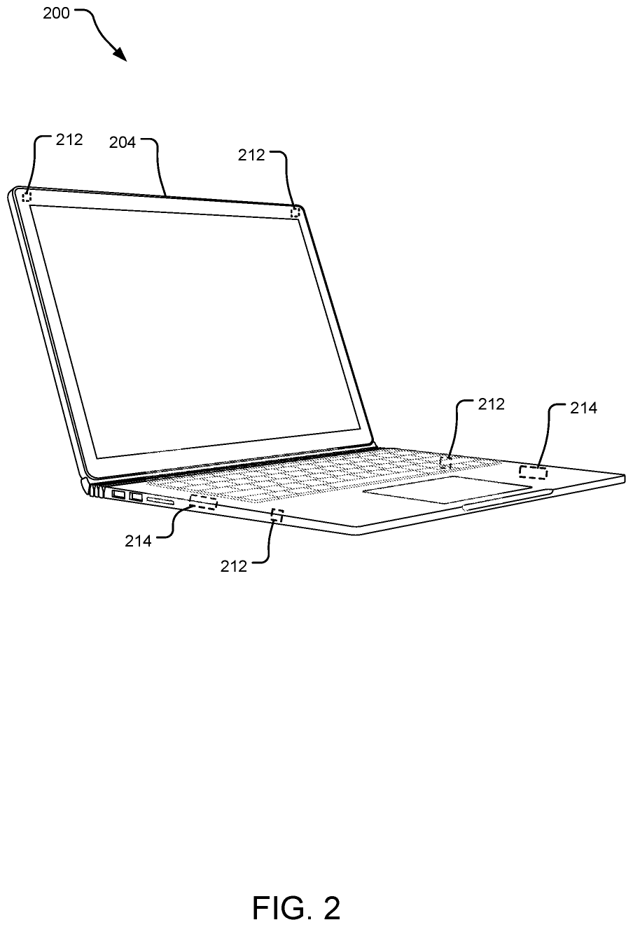

FIG. 2 illustrates an example communication device system 200, which may comprise the communication device 104 of FIG. 1. The communication device 204 is any device capable of communicating wirelessly using electromagnetic radiation, for example, radio frequencies. While the communication device 204 illustrated in FIG. 2 is a laptop, it may be appreciated that any communication device that uses electronic communication is contemplated, for example, a mobile phone, desktop computer, server, tablet, or hybrid 2-in-1 system.

The communication device 204 may have one or more electronic transmitters 212. The one or more electronic transmitters 212 are elements that transmit electronic communications. The electronic transmitters 212 may include one or more of antennas, transceivers, and other communications components and may be configured to transmit in one or more electromagnetic frequency bands. The transmission power for the one or more electronic transmitters 212 can be limited in order to comply with regulatory standards for SAR compliance when a user interacts directly with the communication device 204. For example, the transmission power of the one or more electronic transmitters 212 may be controlled by a power adjustment module in response to classification of detected motion such as described above in the various use cases of FIG. 1. That is, the transmission power supplied to or provided by the electronic transmitters 212 can be increased when a user does not interact directly with the communication device 204.

In the illustrated implementation, the communication device 204 includes one or more motion sensors 214. The one or more motion sensors 214 are elements that detect motion of the communication device 204. It may be appreciated that the one or more motion sensors 214 may be implemented in software, dedicated hardware, or any combination thereof. The motion sensor 214 can therefore be used to discern between SAR motion states in the communication device 204 by classification of detected motion of the communication device 204.

As illustrated, the communication device 204 may include a plurality of motion sensors 214. The motion sensors 214 may be situated or coupled in different parts of the communication device 204, tracking the motion of the respective different parts of the communication device 204. The locations at which the motion sensors 214 are located may correspond to, or be relevant to, one or more electronic transmitters 212 that emit electromagnetic radiation. In implementations, the output of the motion sensors 214 can be individually processed to determine a classification condition and/or can be output collectively to determine a classification condition of detected motion to determine a SAR motion state, perhaps using inferential software, such as machine learning, or applying predefined standards stored in software.

The motion sensor 214 may include any element that detects motion, for example, a sensor that detects acceleration or changes in physical motion (e.g., a gyro, an accelerometer, etc.), a sensor that detects reflections of electromagnetic radiation (e.g., LIDAR, RADAR, etc.), or a sensor that detects reflections of sound (e.g., SONAR). A detected motion can be, for example, one or more of a single sample of detected motion, a motion representing a number of consecutive samples, a pattern of motion within a set of samples or time frame. The motion may be a pattern of motion indicative of a SAR motion state.

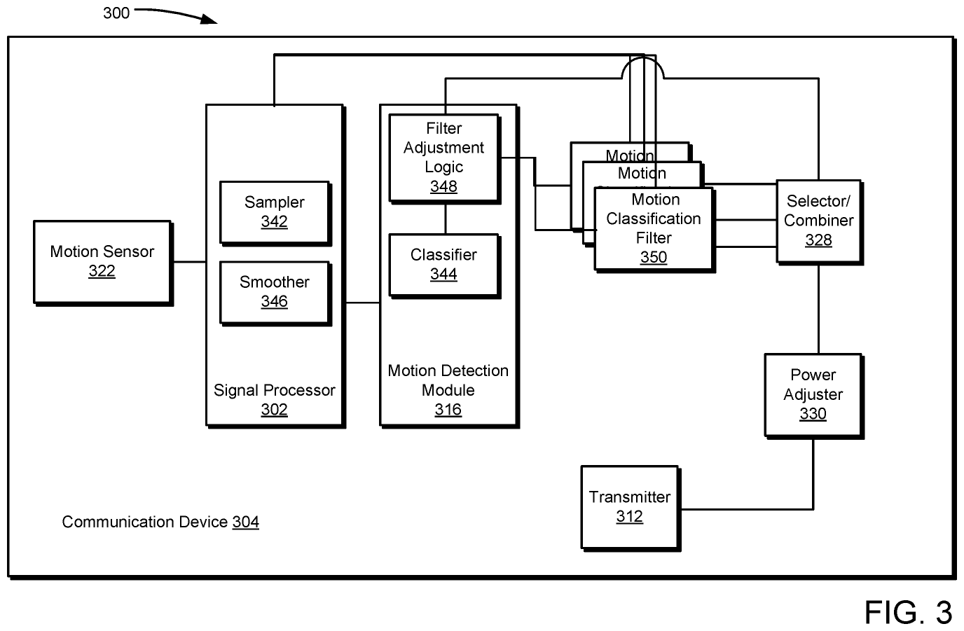

FIG. 3 illustrates a schematic representation 300 of an example communication device 304. The communication device 304 may include a motion sensor 322, a power adjuster 330, and a transmitter 312. The motion sensor 322 may provide a motion signal as an output to a signal processor 302. The signal processor 302 may output a processed signal to a motion detection module 316. The signal processor 302 may comprise signal processing elements, such as one or more of a sampler 342 and a smoother 346. The sampler 342 is an element that samples detected motion to produce a discrete detected motion signal. The smoother 346 is an element that smooths the signal. The smoother may help to reduce the effect of outliers and/or noise in the signal representing the detected motion.

In an example, the smoother 346 may render the signal into an energy representation. The smoother may also use a mean square or root mean square average for smoothing energy representation of consecutive or otherwise related samples. More specifically, the motion sensor 322 may demonstrate detected motion in more than one axis (e.g., x, y, and z axes from a particular reference). A direction-independent magnitude of the energy in the signals for a sample can be determined by taking the square of the signal representing motion detected for the axes and summing them. The smoother 346 may use a mean-squared function or a root-mean-squared function to smooth the direction-independent magnitude of energy in the signal.

The motion detection module 316 may comprise a classifier 344. The classifier 344 may classify one or more of detected motions according to classification conditions by determining the particular type of motion profile to which the detected motion corresponds (e.g., a SAR IN MOTION state or SAR STATIONARY state). The classifier 344 may classify detected motion using any one or more different approaches. For example, the classification of the detected motion may be made in relation to a threshold to which the processed signal representative of the detected motion is compared. Another basis for comparison of whether detected motion represents SAR IN MOTION state or SAR STATIONARY state may be a noise floor that represents motion attributed to elements of the device itself and/or motion characteristic of an environment in which the communication device 304 is present. In some implementations, the magnitude of individual frequency components may be compared to expected motion profiles to determine a likely motion profile that corresponds to the distribution of magnitudes of energy of the signal at different frequencies. For example, when a user interacts with the communication device 304, the motion sensor 322 may be able to detect motions specific to a human body, such as or more of cardiovascular heartbeat, pulse, or other measures of blood flow. The comparison of the motion signal from the motion sensor 322 to a motion profile may be performed by an artificial intelligence module of the classifier 344. In other examples, a frequency domain representation of the motion signal may be used to compare the signal to a motion profile for classification of detected motion by the classifier 344.

The motion detection module 316 may, upon classification of detected motion of the communication device 304, assign one or more motion classification filters 350. The one or more motion classification filters 350 may be executed as hardware, software, firmware, or a combination thereof. The one or more motion classification filters 350 may also receive the motion signal from the signal processor 302. As may be appreciated, the one or more motion classification filters 350 may receive the motion signal from the signal processor 302 without further intervention from the motion detection module 316 once the detected motion has been classified. The one or more motion classification filters 350 may be provided with one or more filter parameters by a filter adjustment logic 348. The filter parameters may be determined by the filter adjustment logic 348 in relation to the classification of the detected motion and/or one or more signal characteristics related to the classified motion. As an example, the classifier 344 may determine that a given amplitude of 1 Hz in the motion signal corresponds to a likely heartbeat of a user in contact with the device. The filter parameters provided to assigned motion classification filters 350 may cause the one or more motion classification filters 350 to monitor a given frequency bandwidth associated with the detected motion to determine if a sufficient amplitude of the signal is present. Other filter parameters including thresholds, decay values, sample window sizes, or the like may also be provided.

Accordingly, the one or more motion classification filters 350 that are assigned and parameterized with the filter parameters from the filter adjustment logic 348 may monitor the motion signal from the signal processor 302 without intervention of the motion detection module 316. In turn, the motion detection module 316 may cease execution during monitoring of the motion signal by the one or more motion classification filters 350. The one or more motion classification filters 350 may continue to monitor the detected motion using the filter parameters until the detected motion is no longer detected by the one or more motion classification filters 350. Upon cessation of the detected motion, the motion detection module 316 may resume execution to classify the resulting changed motion signal and reassign one or more motion classification filters 350 to monitor the new state according to the classified motion (e.g., SAR IN MOTION or SAR STATIONARY)

In one filter implementation, two or more cascaded filters can combine to form any a band pass or a notch filter to allow or eliminate certain frequency components from the monitored signal. Varying the response of each filter will provide more controllable frequency selectivity without the burden of an expensive adaptive filter.

A selector/combiner module 328 may also be configured by the filter adjustment logic 348 to determine which of the one or more motion classification filters 350 are used to determine continued detection of the detected motion. For example, a plurality of the one or more motion classification filters 350 may collectively be used for monitoring of the detected motion. As an example, a bandpass filter arrangement may be provided with given ones of a plurality of the one or more motion classification filters 350 acting as a low pass filter and a high pass filter, respectively, to provide the bandpass filter. Furthermore, a detected motion may correspond to different harmonics of the signal such that different ones of the one or more motion classification filters 350 may be assigned to monitor the signal in relation to the different harmonics at different frequency bandwidths in the signal. In this case, the combiner module 328 may act as an AND gate to determine continued detection of the detected motion based on continued detection by the plurality of motion classification filters 350. The combiner module 328 could apply other logic (e.g., OR gates, NOR gate, XOR gates, or combinations thereof) to monitor a signal characteristic associated with the classified motion as classified by the classifier 344.

In any regard, the combiner module 328 may be in operative communication with the power adjuster 330 to provide an appropriate RF transmission power for the transmitter 312 based on the detected motion. For instance, if the detected motion classified by the classifier 344 and monitored by the one or more motion classification filters 350 continues, the power adjuster 330 may maintain the communication device 304 in a SAR IN MOTION state with a reduction in the transmit power of the transmitter 312. Once such detected motion is discontinued, and the classifier 344 determines that the detected motion correspond to a SAR STATIONARY state (e.g., by expressly classifying the detected motion as such or in the absence of classification of an SAR IN MOTION state), the power adjuster 330 may readjust the power of the transmitter 312 to a higher power setting.

The classifier 344 and/or one or more motion classification filters 350 may also utilize a decay interval for maintaining a given classified motion for a period of time. A decay interval is a period of time or corresponding samples over which a detected state remains triggered based on the detected motion continuing to indicate the given state. The decay interval may require a time frame or a number of samples over which the detected motion is discontinued before the classified detected motion is no longer maintained.

As noted above, the filter adjustment logic 348 may assign filter parameters to the one or more motion classification filters 350 for monitoring a detected motion that has been classified by the classifier 344. As such, the classifier 344 may be in operative communication with the filter adjustment logic 348. The classifier 344 may provide the filter adjustment logic 348 with information regarding the classified motion including, for example, information for determinant appropriate filter parameters to be provided to the one or more motion classification filters 350. As noted above, the filter parameters may include, but are not limited to, a detection threshold value such as a threshold amplitude for the signal energy, a sensitivity value, a variable window average, a frequency of monitored motion, or a detection decay value.

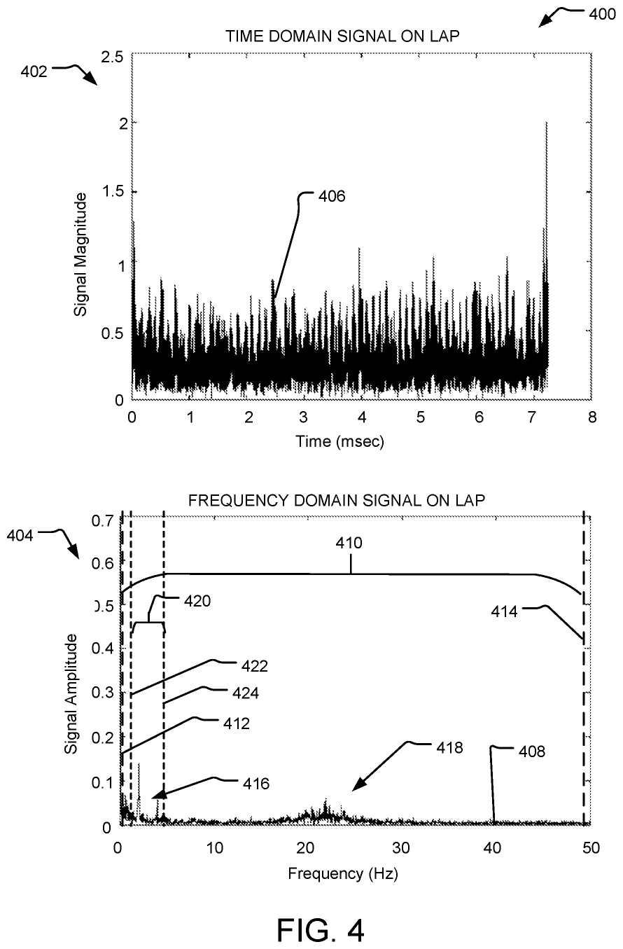

In one example, the filter parameters provided to the one or more motion classification filters 350 may limit the scope of a given signal characteristic monitored by the one or more motion classification filters 350. For example, FIG. 4 includes plots 400 of data corresponding to detected motion from a motion sensor. Plot 402 comprises a time-domain representation of the motion signal 406 magnitude of the motion sensor. That is, the horizontal axis represents time and the vertical axis is the magnitude of the signal energy (e.g., as the summed square of the x, y, and z components of motion).

Plot 404 represents a frequency domain representation of the motion signal 408. As an example, plot 404 is representative of a result of an FFT (e.g., which may be prepared by the classifier 344 of FIG. 3). As may be appreciated, the plot 404 includes frequency on the horizontal axis and signal amplitude on the vertical axis. As such, frequency-based determinations for classification of motion may leverage the frequency domain to detect and classify motion. This may be performed by a classifier over a first range 410 of signal characteristics (e.g., frequency). The first range 410 may span from a first frequency 412 and a second frequency 414. Within the first range 410, there may be identified a first detected movement component 416 at a frequency between around 0 Hz and 5 Hz. In addition, a second detected movement component 418 between 20 Hz and 25 Hz may also be identified. As may be appreciated, a classifier may match the first detected movement component 416 and/or second detected movement component 418 to a motion profile or otherwise classify the movement represented by the first detected movement component 416 and/or second detected movement component 418. For instance, in the example of FIG. 4, a classifier analyzing the motion signal 408 may determine that the first detected movement component 416 corresponds to movement likely associate with the pulse of a user (e.g., based on the device resting on the user such as a lap or the like). The second detected movement component 418 may be determined to correspond to background movement unrelated to the user interaction.

As may be appreciated, use of the frequency domain plot 404 to classify the detected motion may include calculation of an FFT by a classifier, application of an AI model, or other relatively sophisticated processing to classify the first detected movement component 416 or second detected movement component 418. However, once determined that the first detected movement component 416 corresponds to human interaction movement, continued execution of the motion detection module may utilize resources that contribute to computational overhead and/or power consumption. Therefore, the motion detection module may identify the first detected movement component 416 as being the relevant motion for the classification of the movement (e.g., into a SAR IN MOTION state). The motion detection module may also determine relevant filter parameters that bound the signal characteristics for this classified motion for continued detection. For instance, the motion detection module may determine that frequencies in a bandpass in a second range 420 between a third frequency 422 and a fourth frequency 424 are relevant for continued detection of the first detected movement component 416. As may be appreciated, the second range 420 may be a narrower range than the first range 410. As such, upon assignment of the one or more motion classification filters, the filter parameters assigned may correspond to the second range 420 that has been identified by the motion detection module.

For instance, the classifier may determine the first detected movement component 416 corresponds to user-induced motion and identify the one or more filter parameters relevant to the first detected movement component 416. In turn, the filter adjustment logic may provide the identified filter parameter to the one or more motion classification filters for continued monitoring of the first detected movement component 416. Moreover, once the narrowed signal characteristic is identified (e.g., a bandpass for the first detected movement component 416), the benefit of performing the FFT or other advance classification approach may be removed. In turn, the one or more motion classification filters may continue to monitor the first detected movement component 416 from a motion signal of a motion detector without execution of the motion detection module.

As the one or more motion classification filters may be less resource intensive than the motion detection module, the one or more motion classification filters may monitor the first detected movement component 416 until the first detected movement component 416 is no longer detected without the continued execution of the motion detection module. In addition, the filter adjustment logic may set the filter parameters for the one or more motion classification filters such that background motion such as second detected movement component 418 may be disregarded from the monitoring, thus allowing the one or more motion classification filters to monitor activity associated with human interaction.

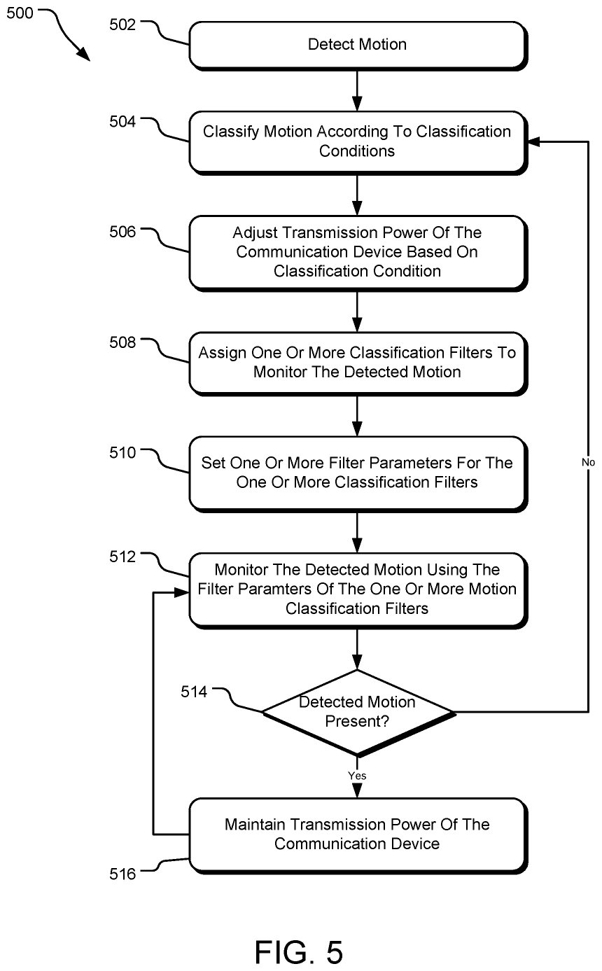

FIG. 5 illustrates example operations 500 for a communication device to classify and monitor detected motion of the communication device. In the example operations 500, the communications device may utilize the classification and monitoring of detected motion in relation to a SAR state of the communication device. However, as noted above, use of motion classification filters as described herein may be generally utilized in connection with classification and monitoring of any motion for identification or performing some action based on the status of that classification. For example, as noted above, mechanical operation of the device may be monitored through detected motion such that classification of the motion of the mechanical operation into states related to malfunction, wear, or other conditions may be classified and monitored generally according to the approach described herein.

The operations 500 may include a detecting operation 502 in which a motion sensor may detect motion of the communication device and output a signal corresponding to the detected motion. A classifying operation 504 may classify the motion according to classification conditions. For example, the classification conditions may correspond to SAR states including, for example, a SAR IN MOTION state or an SAR STATIONARY state as described above. As may be appreciated, the classifying operation 504 may include operation of a motion detection module including a classifier that may apply a classification technique to identify and classify the motion from the motion signal of the detecting operation 502. As such, the classifying operation 504 may include use of classification parameters, an artificial intelligence classification model, an FFT, or other processing technique that may be used to identify or match motion to motion profiles or otherwise classify the motion.

In the specific example operations 500 illustrated, the operations 500 may also include a power adjustment operation 506 in which the transmission power of the communication device may be adjusted based on the classification condition identified by the classifying operation 504. For example, the power adjustment operation 506 may set a transmission power of the communication device to a reduced level when in an SAR IN MOTION state and set the transmission power to an increased level when in a SAR STATIONARY state.

The operations 500 may include a filter assignment operation 508 in which one or more motion classification filters are assigned to monitor the detected motion that was classified in the classifying operation 504. While the one or more motion classification filters may monitor for the classified motion, it may be appreciated that once assigned in the filter assignment operation 508, the motion classification filters may receive the motion signal directly from the motion sensor without continued performance of the classifying operation 504.

In addition, a parameter setting operation 510 may set one or more filter parameters for use by the one or more classification filters for monitoring the detected motion to determine whether the detected motion that was classified in the classifying operation 504 continues. The setting operation 510 may be at least in part based on the classifying operation 504 such that the filter parameters set in the setting operation 510 are related to the nature of the motion that is classified according to the classification condition.

The operations 500 may include a monitoring operation 512 in which the detected motion is monitored using the one or more motion classification filters having the one or more filter parameters. The monitoring operation 512 may exclusively use the one or more motion classification filters such that the motion detection module and classifier that performed the classifying operation 504 may cease execution during the monitoring operation 512.

The operations 500 may include a determination operation 514 in which it is determined whether the detected classified motion continues to be present based on the monitoring operation 512 performed by the one or more motion classification filters 350. If the classified motion continues to be detected by the one or more classification filters during the monitoring operation 512 based on the filter parameters, the operations 500 include a maintaining operation 516 in which the transmission power of the communication device is maintained in the adjusted transmission power setting of the power adjustment operation 506. However, if the detected classified motion monitored using the one or more motion classification filters is no longer detected, the operations 500 may iterate such that the classifying operation 504 may be performed again to classify a different detected motion in a second performance of the classifying operation 504. Correspondingly, the power adjustment operation 506, filter assignment operation 508, parameter setting operation 510, and the monitoring operation 512 may be iteratively performed for different detected motions that are classified in the classifying operation 504. In this regard, classifying operation 504 may be performed upon cessation of the detected motion by the one or more motion classification filters having previously been assigned in a prior iteration of the operations 500.

Additionally or alternatively, the classifying operation 504 may be periodically performed to detect different classified motions. As may be appreciated, given the increased consumption of resources associated with the classifying operation 504, reducing the operation of the classifying operation 504 and relying more on the monitoring operation 512 may improve device performance by utilizing the less resource intensive motion classification filters to monitor the motion having previously been classified and for which filter parameters are set.

In one example of the operations 500, the motion detected in the detecting operation 502 may be classified in the classifying operation 504 as a SAR IN MOTION state associated with user interaction of the device (e.g., including passive interaction determined through motion associated with a user's heartbeat, respiratory movement, or the like). In turn, the power adjustment operation 506 may adjust the transmission power of the communication device to a reduced power level associated with the SAR IN MOTION state. In addition, the filter assignment operation 508, parameter setting operation 510, and monitoring operation 512 may be performed such that the one or more motion classification filters monitor the classified detected motion of the user interaction with the device using the filter parameters set for the one or more motion classification filters 350. The reduced transmission power may be maintained so long as the classified detected motion corresponding to the SAR IN MOTION state is detected by the one or more motion classification filters.

In this example, the one or more motion classification filters may cease to detect classified motion at which time the classifying operation 504 may be repeated. In the repeated classifying operation 504, it may be determined that the detected motion of the communication device now corresponds to a SAR STATIONARY state such that the power adjustment operation 506 may adjust the transmission power of the communication device by increasing the transmission power. The one or more classification filters may continue to monitor the classified detected motion in the monitoring operation 512 using motion classification filters assigned in a subsequent filter assignment operation 508 using filter parameters set to correspond to the motion corresponding to the classified SAR STATIONARY state. The increased transmission power may be maintained so long as the determination operation 514 continues to determine that the monitoring operation 512 confirms continued detection of the classified motion.

Alternatively, upon cessation of the detected motion corresponding to the SAR IN MOTION state, a subsequent iteration of the classifying operation 504 may result in classification of a different type of detected motion may also be classified as a SAR IN MOTION state. In this regard, different motion classification filters may be assigned in the filter assignment operation 508 to monitor the different classified motion. In this regard, in subsequent iterations of the operations 500, the communication device may not alter the power transmission state, but the one or more class motion classification filters may be updated in response to the change in detected motion as identified during the classifying operation 504.

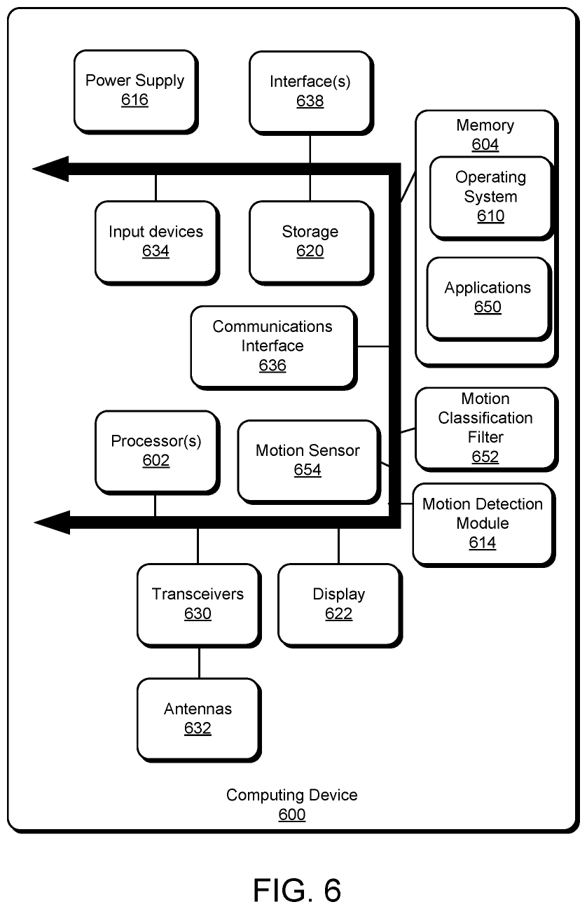

FIG. 6 illustrates an example communication device 600 for implementing the features and operations of the described technology. The communication device 600 may embody a remote-control device or a physical controlled device and is an example network-connected and/or network-capable device and may be a client device, such as a laptop, mobile device, desktop, tablet; a server/cloud device; an internet-of-things device; an electronic accessory; or another electronic device. The communication device 600 includes one or more processor(s) 602 and a memory 604. The memory 604 generally includes both volatile memory (e.g., RAM) and nonvolatile memory (e.g., flash memory). An operating system 610 resides in the memory 604 and is executed by the processor(s) 602. The communication device 600 may be an implementation of communication device 204. The communication device may also have one or more motion sensors 654.

In an example communication device 600, as shown in FIG. 6, one or more modules or segments, such as applications 650, a motion classification filter 652, a motion detection module 614, a SAR management API, a SAR manager, artificial intelligence classifying algorithms, SAR motion frameworks, motion detectors, classifiers, parameter adjusters, samplers, signal discriminators, smoothers, signal processors, sensitivity tuners, controllers, transmission power adjusters, transmitters, and inferential algorithms are loaded into the operating system 610 on the memory 604 and/or storage 620 and executed by processor(s) 602. The storage 620 may include one or more tangible storage media devices and may store SAR motion profiles, SAR motion states, SAR management protocols, SAR instructions, SAR backoff instructions, power transmission adjustment instructions, machine learning algorithms, SAR motion tables, transmission power management tables, classification parameters, classification conditions, controller outputs, thresholds, discriminated frequencies, sample numbers, sensitivity tunings, decay intervals, frequencies indicating user interaction, inferential algorithms, frequencies indicating background motion, locally and globally unique identifiers, requests, responses, and other data and be local to the communication device 600 or may be remote and communicatively connected to the communication device 600.

The communication device 600 includes a power supply 616, which is powered by one or more batteries or other power sources and which provides power to other components of the communication device 600. The power supply 616 may also be connected to an external power source that overrides or recharges the built-in batteries or other power sources.

The communication device 600 may include one or more communication transceivers 630, which may be connected to one or more antenna(s) 632 to provide network connectivity (e.g., mobile phone network, Wi-Fi®, Bluetooth®) to one or more other servers and/or client devices (e.g., mobile devices, desktop computers, or laptop computers). The communication device 600 may further include a network adapter 636, which is a type of communication device. The communication device 600 may use the adapter and any other types of communication devices for establishing connections over a wide-area network (WAN) or local-area network (LAN). It may be appreciated that the network connections shown are examples and that other communication devices and means for establishing a communications link between the communication device 600 and other devices may be used.

The communication device 600 may include one or more input devices 634 such that a user may enter commands and information (e.g., a keyboard or mouse). These and other input devices may be coupled to the server by one or more interfaces 638, such as a serial port interface, parallel port, or universal serial bus (USB). The communication device 600 may further include a display 622, such as a touch screen display.

The communication device 600 may include a variety of tangible processor-readable storage media and intangible processor-readable communication signals. Tangible processor-readable storage can be embodied by any available media that can be accessed by the communication device 600 and includes both volatile and nonvolatile storage media, removable and non-removable storage media. Tangible processor-readable storage media excludes communications signals (e.g., signals per se) and includes volatile and nonvolatile, removable and non-removable storage media implemented in any method or technology for storage of information such as processor-readable instructions, data structures, program modules, or other data. Tangible processor-readable storage media includes, but is not limited to, RAM, ROM, EEPROM, flash memory or other memory technology, CDROM, digital versatile disks (DVD) or other optical disk storage, magnetic cassettes, magnetic tape, magnetic disk storage or other magnetic storage devices, or any other tangible medium which can be used to store the desired information and which can be accessed by the communication device 600. In contrast to tangible processor-readable storage media, intangible processor-readable communication signals may embody processor-readable instructions, data structures, program modules, or other data resident in a modulated data signal, such as a carrier wave or other signal transport mechanism. The term “modulated data signal” means a signal that has one or more of its characteristics set or changed in such a manner as to encode information in the signal. By way of example, and not limitation, intangible communication signals include signals traveling through wired media such as a wired network or direct-wired connection, and wireless media such as acoustic, RF, infrared, and other wireless media.

Various software components described herein are executable by one or more processors, which may include logic machines configured to execute hardware or firmware instructions. For example, the processors may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

Aspects of processors and storage may be integrated together into one or more hardware logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of a remote-control device and/or a physical controlled device implemented to perform a particular function. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

It will be appreciated that a “service,” as used herein, is an application program executable across one or multiple user sessions. A service may be available to one or more system components, programs, and/or other services. In some implementations, a service may run on one or more server computing devices.

The logical operations making up embodiments described herein may be referred to variously as operations, steps, objects, or modules. Furthermore, it should be understood that logical operations may be performed in any order, adding or omitting operations as desired, regardless of whether operations are labeled or identified as optional, unless explicitly claimed otherwise or a specific order is inherently necessitated by the claim language. While this specification contains many specific implementation details, these should not be construed as limitations on the scope of any inventions or of what may be claimed, but rather as descriptions of features specific to particular implementations of the particular described technology. Certain features that are described in this specification in the context of separate implementations can also be implemented in combination in a single implementation. Conversely, various features that are described in the context of a single implementation can also be implemented in multiple implementations separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

In some aspects, the techniques described herein relate to a method of monitoring detected motion of a communication device, including: detecting motion of the communication device using a motion sensor; classifying detected motion according to one or more classification conditions; assigning one or more motion classification filters to monitor the detected motion; setting one or more filter parameters for the one or more motion classification filters; and monitoring the detected motion using the one or more filter parameters.

In some aspects, the techniques described herein relate to a method, further including: adjusting transmission power of the communication device responsive to continued detection of the detected motion by the one or more motion classification filters using the one or more filter parameters.

In some aspects, the techniques described herein relate to a method, further including: detecting, by the one or more motion classification filters, a discontinuation of the detected motion based on the one or more filter parameters; and classifying different detected motion using the motion sensor responsive to detecting the discontinuation.

In some aspects, the techniques described herein relate to a method, further including: readjusting the transmission power of the communication device responsive to a failure to detect the different detected motion.

In some aspects, the techniques described herein relate to a method, further including: classifying the different detected motion according to the one or more classification conditions; assigning one or more different motion classification filters to monitor the different detected motion; setting one or more different filter parameters for the one or more different motion classification filters; monitoring the different detected motion using the one or more different filter parameters; and adjusting the transmission power of the communication device responsive to continued detection of the different detected motion by the one or more different motion classification filters using the one or more different filter parameters.

In some aspects, the techniques described herein relate to a method, wherein the classifying the detected motion includes analyzing a first range of signal characteristics, and wherein the one or more filter parameters for the one or more motion classification filters limit the monitoring the detected motion using the one or more filter parameters to a second range of signal characteristics narrower than the first range of signal characteristics.

In some aspects, the techniques described herein relate to a method, wherein the classifying the detected motion includes application of motion detection model by a motion detection module, and wherein the motion detection module discontinues execution during the monitoring the detected motion using the one or more filter parameters.

In some aspects, the techniques described herein relate to a system having a processor and a memory for monitoring detected motion of a communication device of a communication device, including: a motion sensor to detect motion of the communication device; a motion detection module executable by the processor to classify detected motion according to one or more motion classification conditions; and one or more motion classification filters to monitor the detected motion using one or more filter parameters set for the one or more motion classification filters by the motion detection module.

In some aspects, the techniques described herein relate to a system, further including: a transmission power adjuster executable by the processor to adjust transmission power of the communication device based at least in part on continued detection of the detected motion by the one or more motion classification filters.

In some aspects, the techniques described herein relate to a system, wherein: the one or more motion classification filters detects a discontinuation of the detected motion based on the one or more filter parameters; and the motion detection module classifies different detected motion from the motion sensor responsive to the discontinuation.

In some aspects, the techniques described herein relate to a system, wherein the transmission power adjuster readjusts the transmission power of the communication device responsive to a failure to detect the different detected motion.

In some aspects, the techniques described herein relate to a system, wherein: the motion detection module classifies different detected motion using the motion sensor responsive to the discontinuation to classify the different detected motion according to the one or more motion classification conditions, assign one or more different motion classification filters to monitor the different detected motion, and set one or more different filter parameters for the one or more different motion classification filters; wherein the one or more motion classification filters monitors the different detected motion using the one or more different filter parameters, and the transmission power adjuster adjusts the transmission power of the communication device based at least in part on continued detection of the detected motion by the one or more motion classification filters.

In some aspects, the techniques described herein relate to a system, wherein the motion detection module analyzes a first range of signal characteristics to classify detected motion, and wherein the one or more filter parameters for the one or more motion classification filters limit the one or more motion classification filters to a second range of signal characteristics narrower than the first range of signal characteristics.

In some aspects, the techniques described herein relate to a system, wherein the motion detection module discontinues execution during the monitoring of the detected motion by the one or more motion classification filters.

In some aspects, the techniques described herein relate to one or more tangible processor-readable storage media embodied with instructions for executing on one or more processors and circuits a process for monitoring detected motion of a communication device, including: detecting motion of the communication device using a motion sensor; classifying detected motion according to one or more classification conditions; assigning one or more motion classification filters to monitor the detected motion; setting one or more filter parameters for the one or more motion classification filters; and monitoring the detected motion using the one or more filter parameters.

In some aspects, the techniques described herein relate to one or more tangible processor-readable storage media, wherein the process further includes: adjusting transmission power of the communication device responsive to continued detection of the detected motion by the one or more motion classification filters using the one or more filter parameters.

In some aspects, the techniques described herein relate to one or more tangible processor-readable storage media, wherein the process further includes: detecting, by the one or more motion classification filters, a discontinuation of the detected motion based on the one or more filter parameters; and classifying different detected motion using the motion sensor responsive to detecting the discontinuation.

In some aspects, the techniques described herein relate to one or more tangible processor-readable storage media, wherein the process further includes: readjusting the transmission power of the communication device responsive to a failure to detect the different detected motion.

In some aspects, the techniques described herein relate to one or more tangible processor-readable storage media, wherein the process further includes: classifying the different detected motion according to the one or more classification conditions; assigning one or more different motion classification filters to monitor the different detected motion; setting one or more different filter parameters for the one or more different motion classification filters; monitoring the different detected motion using the one or more different filter parameters; and adjusting the transmission power of the communication device responsive to continued detection of the different detected motion by the one or more different motion classification filters using the one or more different filter parameters.

In some aspects, the techniques described herein relate to one or more tangible processor-readable storage media, wherein the classifying the detected motion includes analyzing a first range of signal characteristics, and wherein the one or more filter parameters for the one or more motion classification filters limit the monitoring the detected motion using the one or more filter parameters to a second range of signal characteristics narrower than the first range of signal characteristics.

In some aspects, the techniques described herein relate to a system for monitoring detected motion of a communication device, including: means for detecting motion of the communication device using a motion sensor; means for classifying detected motion according to one or more classification conditions; means for assigning one or more motion classification filters to monitor the detected motion; means for setting one or more filter parameters for the one or more motion classification filters; and means for monitoring the detected motion using the one or more filter parameters.

In some aspects, the techniques described herein relate to a system, further including: means for adjusting transmission power of the communication device responsive to continued detection of the detected motion by the one or more motion classification filters using the one or more filter parameters.

In some aspects, the techniques described herein relate to a system, further including: means for detecting, by the one or more motion classification filters, a discontinuation of the detected motion based on the one or more filter parameters; and means for classifying different detected motion using the motion sensor responsive to detecting the discontinuation.

In some aspects, the techniques described herein relate to a system, further including: means for readjusting the transmission power of the communication device responsive to a failure to detect the different detected motion.

In some aspects, the techniques described herein relate to a system, further including: means for classifying the different detected motion according to the one or more classification conditions; means for assigning one or more different motion classification filters to monitor the different detected motion; means for setting one or more different filter parameters for the one or more different motion classification filters; means for monitoring the different detected motion using the one or more different filter parameters; and means for adjusting the transmission power of the communication device responsive to continued detection of the different detected motion by the one or more different motion classification filters using the one or more different filter parameters.

In some aspects, the techniques described herein relate to a system, wherein the means for classifying the detected motion includes analyzing a first range of signal characteristics, and wherein the one or more filter parameters for the one or more motion classification filters limit the monitoring the detected motion using the one or more filter parameters to a second range of signal characteristics narrower than the first range of signal characteristics.

In some aspects, the techniques described herein relate to a system, wherein the means for classifying the detected motion includes application of motion detection model by a motion detection module, and wherein the motion detection module discontinues execution during the monitoring the detected motion using the one or more filter parameters.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

A number of implementations of the described technology have been described. Nevertheless, it will be understood that various modifications can be made without departing from the spirit and scope of the recited claims.

What is claimed is:

1. A method of monitoring detected motion of a communication device, comprising:
detecting motion of the communication device using a motion sensor;
classifying detected motion according to one or more classification conditions;
assigning one or more motion classification filters to monitor the detected motion;
setting one or more filter parameters for the one or more motion classification filters; and
monitoring the detected motion using the one or more filter parameters.

2. The method of claim 1, further comprising:
adjusting transmission power of the communication device responsive to continued detection of the detected motion by the one or more motion classification filters using the one or more filter parameters.

3. The method of claim 2, further comprising:
detecting, by the one or more motion classification filters, a discontinuation of the detected motion based on the one or more filter parameters; and
classifying different detected motion using the motion sensor responsive to detecting the discontinuation.

4. The method of claim 3, further comprising:
readjusting the transmission power of the communication device responsive to a failure to detect the different detected motion.

5. The method of claim 3, further comprising:
classifying the different detected motion according to the one or more classification conditions;
assigning one or more different motion classification filters to monitor the different detected motion;
setting one or more different filter parameters for the one or more different motion classification filters;
monitoring the different detected motion using the one or more different filter parameters; and
adjusting the transmission power of the communication device responsive to continued detection of the different detected motion by the one or more different motion classification filters using the one or more different filter parameters.

6. The method of claim 1, wherein the classifying the detected motion comprises analyzing a first range of signal characteristics, and wherein the one or more filter parameters for the one or more motion classification filters limit the monitoring the detected motion using the one or more filter parameters to a second range of signal characteristics narrower than the first range of signal characteristics.

7. The method of claim 1, wherein the classifying the detected motion comprises application of motion detection model by a motion detection module, and wherein the motion detection module discontinues execution during the monitoring the detected motion using the one or more filter parameters.

8. A system having a processor and a memory for monitoring detected motion of a communication device of a communication device, comprising:
a motion sensor to detect motion of the communication device;
a motion detection module executable by the processor to classify detected motion according to one or more motion classification conditions; and
one or more motion classification filters to monitor the detected motion using one or more filter parameters set for the one or more motion classification filters by the motion detection module.

9. The system of claim 8, further comprising:
a transmission power adjuster executable by the processor to adjust transmission power of the communication device based at least in part on continued detection of the detected motion by the one or more motion classification filters.

10. The system of claim 9, wherein:
the one or more motion classification filters detects a discontinuation of the detected motion based on the one or more filter parameters; and
the motion detection module classifies different detected motion from the motion sensor responsive to the discontinuation.

11. The system of claim 10, wherein the transmission power adjuster readjusts the transmission power of the communication device responsive to a failure to detect the different detected motion.

12. The system of claim 10, wherein:
the motion detection module classifies different detected motion using the motion sensor responsive to the discontinuation to classify the different detected motion according to the one or more motion classification conditions, assign one or more different motion classification filters to monitor the different detected motion, and set one or more different filter parameters for the one or more different motion classification filters;
wherein the one or more motion classification filters monitors the different detected motion using the one or more different filter parameters, and the transmission power adjuster adjusts the transmission power of the communication device based at least in part on continued detection of the detected motion by the one or more motion classification filters.

13. The system of claim 8, wherein the motion detection module analyzes a first range of signal characteristics to classify detected motion, and wherein the one or more filter parameters for the one or more motion classification filters limit the one or more motion classification filters to a second range of signal characteristics narrower than the first range of signal characteristics.

14. The system of claim 8, wherein the motion detection module discontinues execution during the monitoring of the detected motion by the one or more motion classification filters.

15. One or more tangible processor-readable storage media embodied with instructions for executing on one or more processors and circuits a process for monitoring detected motion of a communication device, comprising:
detecting motion of the communication device using a motion sensor;
classifying detected motion according to one or more classification conditions;
assigning one or more motion classification filters to monitor the detected motion;
setting one or more filter parameters for the one or more motion classification filters; and
monitoring the detected motion using the one or more filter parameters.

16. The one or more tangible processor-readable storage media of claim 15, wherein the process further comprises:
adjusting transmission power of the communication device responsive to continued detection of the detected motion by the one or more motion classification filters using the one or more filter parameters.

17. The one or more tangible processor-readable storage media of claim 16, wherein the process further comprises:
detecting, by the one or more motion classification filters, a discontinuation of the detected motion based on the one or more filter parameters; and
classifying different detected motion using the motion sensor responsive to detecting the discontinuation.

18. The one or more tangible processor-readable storage media of claim 17, wherein the process further comprises:
readjusting the transmission power of the communication device responsive to a failure to detect the different detected motion.

19. The one or more tangible processor-readable storage media of claim 17, wherein the process further comprises:
classifying the different detected motion according to the one or more classification conditions;
assigning one or more different motion classification filters to monitor the different detected motion;
setting one or more different filter parameters for the one or more different motion classification filters;
monitoring the different detected motion using the one or more different filter parameters; and
adjusting the transmission power of the communication device responsive to continued detection of the different detected motion by the one or more different motion classification filters using the one or more different filter parameters.

20. The one or more tangible processor-readable storage media of claim 15, wherein the classifying the detected motion comprises analyzing a first range of signal characteristics, and wherein the one or more filter parameters for the one or more motion classification filters limit the monitoring the detected motion using the one or more filter parameters to a second range of signal characteristics narrower than the first range of signal characteristics.
